(19) United States
(12) Patent Application Publication
(10) Pub. No.: US 2017/0327563 A1
(43) Pub. Date: Nov. 16, 2017

(54) Methods for Treating HIV-Associated Enteropathy Symptoms With Immunoglobulin/Protein Isolate

(71) Applicant: THE LAURIDSEN GROUP, INC., ANKENY, IA (US)

(72) Inventors: Eric Weaver, Ankeny, IA (US); Gerald Klein, Cary, NC (US); Audrey L. Shaw, Cary, NC (US)

(73) Assignee: THE LAURIDSEN GROUP, INC., ANKENY, IA (US)

(21) Appl. No.: 15/151,366

(22) Filed: May 10, 2016

(51) Int. Cl. C07K 16/06; A61K 39/395; A61K 35/16; C12Q 1/70; A61K 9/00

(52) U.S. Cl. CPC C07K 16/06; C12Q 1/703; A61K 9/0053; A61K 35/16; A61K 39/39516

(57) ABSTRACT

The present invention provides a method for treating human immunodeficiency virus (HIV)-associated enteropathy in subjects who have been on antiretroviral therapy by orally administering immunoglobulin/protein isolate.

### FIELD OF THE INVENTION

The present invention relates to methods for treating HIV-associated enteropathy. In particular, this invention relates to methods that comprise administering immunoglobulin/protein isolate from an animal source, such as serum-derived bovine immunoglobulin/protein isolate (SBI).

### BACKGROUND OF THE INVENTION

Noninfectious diarrhea continues to be a problem for nearly 30% of patients infected with the human immunodeficiency virus (HIV) despite control of viral replication while on antiretroviral therapy (ART). Siddiqui, U. et al., 41 J. CLIN. GASTROENTEROL. 484-490 (2007). HIV-associated enteropathy may be the direct result of HIV infection of cells in the intestinal tract, including gut-associated lymphoid tissue (GALT). Without being bound by theory, HIV-associated enteropathy is thought to be caused by ongoing inflammation, mucosal damage, imbalances in gut microbiota, or a combination of these factors. MacArthur, R. D. et al., 55 CLIN. INFECT. DIS. 860-867 (2012). Despite effective viral suppression with ART, some HIV patients continue to show low CD4⁺ counts and/or impaired CD4⁺ functional activity (peripheral and GALT). Dillon, S. M. et al., 7 MUCOSAL IMMUNOLOGY 983-994 (2014). Such patients also have a typically poorer clinical outlook. Thus, there exists a need for treating HIV-associated enteropathy in a subset of HIV patients who have been on ART for a substantial period of time.

### SUMMARY OF THE INVENTION

Surprisingly, the present inventors discovered a method of treating HIV-associated enteropathy in a subject, comprising (1) identifying a subject who has a baseline HIV viral load of from about 20 to about 100 copies/mL and has been on ART for at least about 48 weeks, and (2) orally administering to the subject a therapeutically effective amount of immunoglobulin/protein isolate from an animal source other than human, such as SBI, wherein the immunoglobulin/protein isolate contains at least about 60% by weight Ig, and uses related thereto.

In one embodiment of the invention, the immunoglobulin/protein isolate contains from about 60% to about 80% by weight Ig. In a further embodiment, the immunoglobulin/protein isolate contains greater than about 50% by weight IgG. In yet a further embodiment, the immunoglobulin/protein isolate contains greater than about 50% by weight IgG and less than about 80% by weight IgG. In another embodiment of the invention, the immunoglobulin/protein isolate contains from about 1% to about 10% by weight IgA. In a further embodiment, the immunoglobulin/protein isolate contains about 1% by weight IgA. In another embodiment of the invention, the immunoglobulin/protein isolate contains from about 1% to about 10% by weight IgM. In a further embodiment, the immunoglobulin/protein isolate contains about 5% by weight IgM. In another embodiment of the invention, the immunoglobulin/protein isolate contains from about 1% to about 10% by weight albumin. In a further embodiment, the immunoglobulin/protein isolate contains about 5% albumin.

In one embodiment of the invention, the subject has a baseline HIV viral load of from about 20 to about 40 copies/mL. In another embodiment of the invention, the subject has a peripheral CD4⁺ T cell count of less than about 500 cells/mm³ at baseline. In a further embodiment, the subject has a peripheral CD4⁺ T cell count of from about 300 to about 400 cells/mm³ at baseline.

In one embodiment of the invention, from about 0.5 g to about 15 g of immunoglobulin/protein isolate is administered to the subject daily. In another embodiment of the invention, about 2.5 g of immunoglobulin/protein isolate is administered to the subject twice daily. In a further embodiment, about 5.0 g of immunoglobulin/protein isolate is administered to the subject twice daily. In another embodiment of the invention, the amount of immunoglobulin/protein isolate administered is from about 0.05% to about 5% of the subject's total dietary intake. In a further embodiment, the amount of immunoglobulin/protein isolate administered is about 0.2% of the subject's total dietary intake. In a further embodiment, the amount of immunoglobulin/protein isolate administered is about 0.4% of the subject's total dietary intake.

In one embodiment of the invention, the immunoglobulin/protein isolate is administered for about 1 week to about 25 weeks. In a further embodiment, the immunoglobulin/protein isolate is administered for about 1 week. In a further embodiment, the immunoglobulin/protein isolate is administered for about 2 weeks. In a further embodiment, the immunoglobulin/protein isolate is administered for about 3 weeks. In a further embodiment, the SBI is administered for about 4 weeks. In a further embodiment, the immunoglobulin/protein isolate is administered for about 24 weeks.

In one embodiment of the invention, the subject's peripheral CD4⁺ T cell count increases by at least about 10%. In one embodiment of the invention, the subject's mucosal CD4⁺ lymphocyte density increases by at least about 50%. In one embodiment of the invention, the CD3⁺:CD4⁺ T cell ratio in the duodenal tissue of the subject increases by at least about 50%. In one embodiment of the invention, the plasma IL-6 levels in the subject decreases by at least about 50%. In one embodiment of the invention, the duodenal crypt cells expressing Ki67 in the subject decrease by at least about 50%. In one embodiment of the invention, the CD4⁺:CD8⁺ T cell ratio in the duodenal tissue of the subject increases by at least about 50%.

### BRIEF DESCRIPTION OF THE DRAWING FIGURES

FIG. 1 illustrates the clinical trial design and schedule of outcome procedure for a dual-phase study of SBI administration in patients diagnosed with HIV-associated enteropathy.

FIG. 2 provides the details of the patient population recruited for the dual-phase study of SBI administration in patients diagnosed with HIV-associated enteropathy. The total study population included 103 patients.

FIG. 3 illustrates the peripheral CD4⁺ T cell counts (cells/mm³) for all study patients who received SBI therapy at baseline, and weeks 4 and 24 of the study. The peripheral CD4⁺ T cell counts significantly improved for those study patients who received SBI therapy and who also had the lowest baseline CD4⁺ T cell counts (e.g., 189-418 cells/mm³).

FIG. 4 illustrates the increase in peripheral CD4⁺ T cell counts (cells/mm³) for study patients who were in the lowest baseline CD4⁺ T cell quartile and who also received SBI therapy.

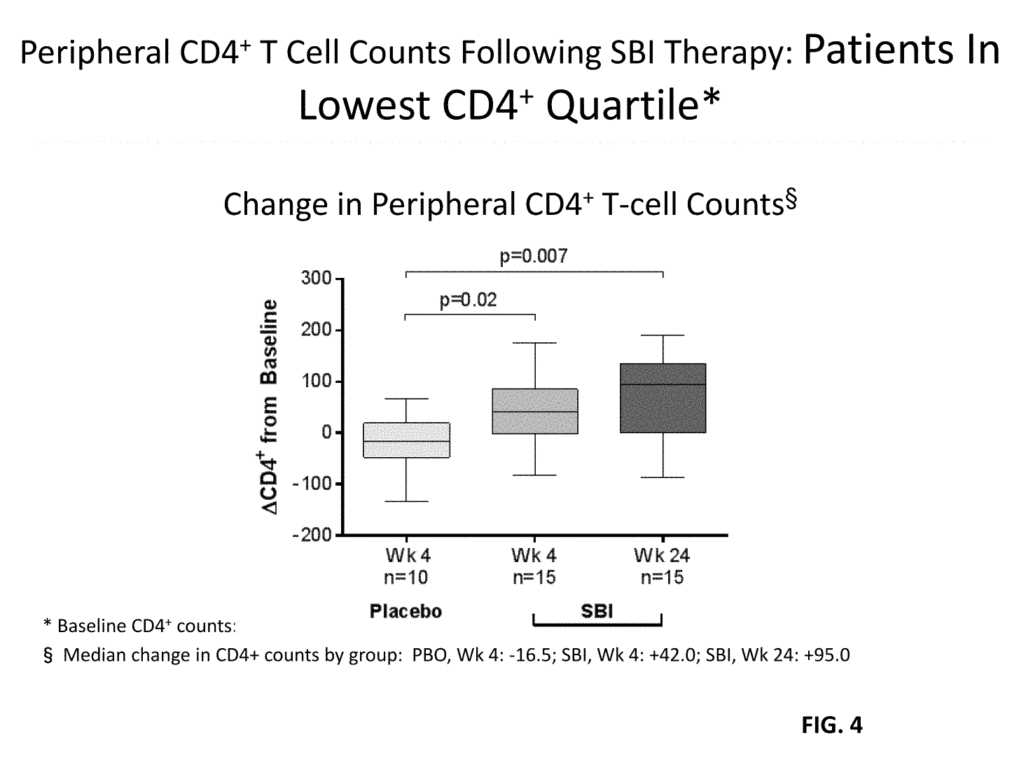

FIG. 5 illustrates the change in duodenal gut-associated lymphoid tissue (GALT) CD4⁺ T cell densities in subset of study patients who received SBI therapy, as measured at baseline and at week 24 (n=8). Duodenal GALT CD4⁺ T cell densities increased from about 217 to about 329 cells/mm², with a median increase of 145 cells/mm² (p=0.02).

FIG. 6 illustrates the change in duodenal CD4⁺:CD8⁺ T cell ratios in a subset of study patients who received SBI therapy, as measured at baseline and at week 24 (n=7). CD4⁺:CD8⁺ T cell ratios increased from a median of about 0.38 to about 0.76 (p=0.0078).

FIG. 7 illustrates the decrease in proliferation of epithelial cells in duodenal tissue in a subset of study patients who received SBI therapy, as measured at baseline and at week 24. (A) Duodenal crypt cells expressing Ki67 decreased in 7 patients from a median of about 40.5% to about 24.4% (p=0.08). (B) Change in Paneth cells in duodenal tissue decreased in 8 patients from a median of about 4.6 Paneth cells/crypt to about 2.8 Paneth cells/crypt (p=0.008).

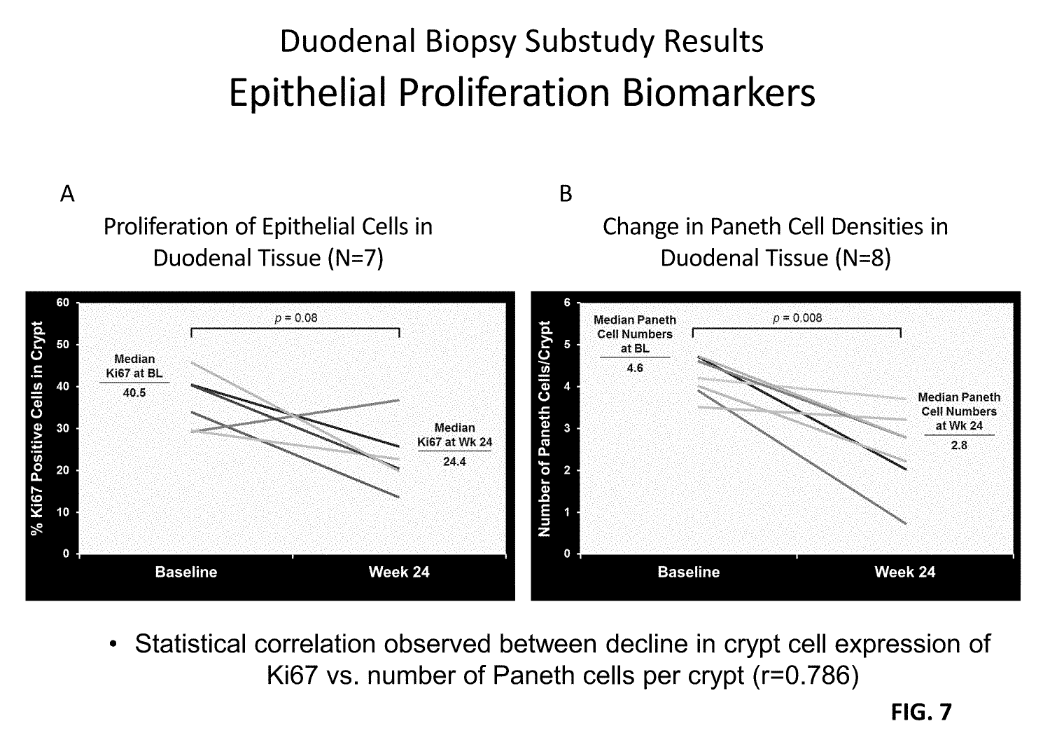

### DETAILED DESCRIPTION OF THE INVENTION

### Definitions

Embodiments may be practiced without the theoretical aspects presented. Moreover, the theoretical aspects are presented with the understanding that the embodiments are not bound by any theory presented.

Unless otherwise defined, all terms (including technical and scientific terms) used herein have the same meaning as commonly understood by one of ordinary skill in the art. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The terminology used herein is for the purpose of describing particular embodiments only and is not intended to be limiting. As used herein, the singular forms “a”, “an” and “the” are intended to include plural forms as well, unless the context clearly indicates otherwise. Thus, for example, reference to “a dose” or “the dose” also includes a plurality of doses. Additionally, as used herein, the term “comprises” is intended to indicate a non-exhaustive list of components or steps, thus indicating that the given composition or method includes the listed components or steps and may also include additional components or steps not specifically listed. As an example, a core weight “comprising SBI” may also include additional components, such as flavorants, colorants, etc. The term “comprising” is also intended to encompass embodiments “consisting essentially of” and “consisting of” the listed components or steps. Similarly, the term “consisting essentially of” is also intended to encompass embodiments “consisting of” the listed components or steps. Furthermore, to the extent that the terms “including”, “includes”, “having”, “has”, “with”, or variants thereof are used in either the detailed description and/or the claims, such terms are intended to be inclusive in a manner similar to the term “comprising.”

Recitation of ranges of values herein are merely intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of ±10% of the referenced value. In other embodiments, the term “about” indicates that the number differs from the given number by less than 9%, 8%, 7%, 6%, 5%, 4%, 3%, 2%, or 1%.

### Immunoglobulin/Protein Isolate

The present disclosure recites methods that involve administration of immunoglobulin/protein isolate from an animal source other than human. Immunoglobulin/protein isolate is a composition purified from animal sources including blood, egg, or milk-which retains the Fc region of the immunoglobulin molecule. Serum-derived immunoglobulin/protein isolate is a protein isolate derived from whole blood, plasma, or serum of a non-human animal that consists of immunoglobulins (Ig) with broad specificity against bacterial and viral antigens. The immunoglobulin/protein isolate can be obtained from whole blood, plasma, or serum from any suitable animal source other than human. Because of the relative availability of its starting material (e.g., whole blood), particularly useful animal sources include bovine (SBI), ovine (SOD, or porcine (SPI) serum. As one example, SBI is a medical food that can be administered orally, under physician supervision, with an excellent safety profile with FDA Generally Regarded As Safe (GRAS) status.

As used herein with reference to the composition used in the invention, the terms “globulin concentrate,” “immunoglobulin concentrate,” “globulin isolate,” “immunoglobulin isolate,” and “immunoglobulin/protein isolate” may be used interchangeably and are intended to describe a composition, purified from animal blood, plasma, serum, egg, or milk sources, that contains relatively high levels of immunoglobulin molecules, wherein the immunoglobulin molecules substantially retain the Fc region. This includes transgenic recombinant immunoglobulins purified from transgenic bacteria, plants or animals. One example of commercially-available globulin concentrate is ImmunoLin™ available from Proliant Inc. Another source of immunoglobulin is EnteraGam® available from EnteraHealth Inc. EnteraGam® is derived from purified bovine serum. EnteraGam® contains immunoglobulin G (IgG), other immunoglobulins, albumin, and transferrin.

Globulin concentrate may be purified according to any of a number of methods available in the art, including those described in Steinbuch, M. and R. Audran, 134 ARCH. BIOCHEM. BIOPHYS. 279-284 (1969); Lee, Y. T. et al., 35 J. AGR. FOOD CHEM. 958-962 (1987); and Polson, A. et al., 82 BIOCHEM. BIOPHYS. ACTA. 463-475 (1964). For example, globulin concentrates can be obtained by spray drying, lyophilization, or any other drying method, and the concentrates may be used in their liquid or frozen form. The active ingredient may also be microencapsulated, protecting and stabilizing from high temperature, oxidants, pH-like humidity, etc. The pharmaceutical compositions used in the invention can be in tablets, capsules, ampoules for oral use, granulate powder, cream, both as a unique ingredient and associated with other excipients or active compounds, or even as a food additive.

One method of obtaining a globulin concentrate used in the invention uses whole blood from a non-human animal as a starting material. Anticoagulant is added to whole blood and then the blood is centrifuged to separate the plasma. Any anticoagulant may be used for this purpose, including sodium citrate and heparin. Persons skilled in the art can readily appreciate such anticoagulants. Calcium is then added to the plasma to promote clotting, the conversion of fibrinogen to fibrin; however other methods are acceptable. This mixture is then centrifuged to remove the fibrin portion from the plasma, resulting in serum. Material is initially fractionated by a modified Cohn procedure and further isolated from other plasma proteins by extensive membrane dialysis and filtration.

The final immunoglobulin/protein isolate can optionally be spray-dried into a powder. The powder allows for easier packaging and the product remains stable for a longer period of time than the raw globulin concentrate in liquid or frozen form.

In one embodiment of the invention, the immunoglobulin/protein isolate contains at least about 60% by weight Ig. In other embodiments, the immunoglobulin/protein isolate contains from about 60% to about 80% by weight Ig. In further embodiments, the immunoglobulin/protein isolate contains from about 60% to about 65%, from about 60% to about 70%, from about 60% to about 75%, from about 60% to about 80%, from about 65% to about 70%, from about 65% to about 75%, from about 65% to about 80%, from about 70% to about 75%, or from about 70% to about 80% by weight Ig. In other embodiments of the invention, the immunoglobulin/protein isolate contains about 60%, 61%, 62%, 63%, 64%, 65%, 66%, 67%, 68%, 69%, 70%, 71%, 72%, 73%, 74%, 75%, 76%, 77%, 78%, 79% or 80% by weight Ig.

In one embodiment of the invention, the immunoglobulin/protein isolate contains greater than about 50% and less than about 80% by weight IgG. In further embodiments, the immunoglobulin/protein isolate contains from about 55% to about 60%, from about 55% to about 65%, from about 55% to about 70%, from about 55% to about 75%, from about 60% to about 65%, from about 60% to about 70%, from about 60% to about 75%, from about 65% to about 70%, from about 65% to about 75% by weight IgG. In other embodiments of the invention, the immunoglobulin/protein isolate contains about 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, or 79% by weight IgG.

In one embodiment of the invention, the immunoglobulin/protein isolate contains from about 1% to about 10% by weight IgA. In further embodiments, the immunoglobulin/protein isolate contains from about 1% to about 2%, from about 1% to about 3%, from about 1% to about 4%, from about 1% to about 5%, from about 1% to about 6%, from about 1% to about 7%, from about 1% to about 8%, from about 1% to about 9%, from about 2% to about 4%, from about 2% to about 6%, from about 2% to about 8%, from about 5% to about 10%, from about 3% to about 6%, from about 3% to about 9%, or from about 8% to about 10% by weight IgA. In other embodiments of the invention, the immunoglobulin/protein isolate contains about 1%, 2%, 3%, 4%, 5%, 6%, 7%, 8%, 9% or 10% by weight IgA. In a further embodiment, the immunoglobulin/protein isolate contains about 1% by weight IgA.

In one embodiment of the invention, the immunoglobulin/protein isolate contains from about 1% to about 10% by weight IgM. In further embodiments the immunoglobulin/protein isolate contains from about 1% to about 2%, from about 1% to about 3%, from about 1% to about 4%, from about 1% to about 5%, from about 1% to about 6%, from about 1% to about 7%, from about 1% to about 8%, from about 1% to about 9%, from about 2% to about 4%, from about 2% to about 6%, from about 2% to about 8%, from about 5% to about 10%, from about 3% to about 6%, from about 3% to about 9%, or from about 8% to about 10% by weight IgM. In other embodiments of the invention, the immunoglobulin/protein isolate contains about 1%, 2%, 3%, 4%, 5%, 6%, 7%, 8%, 9% or 10% by weight IgM. In a further embodiment, the immunoglobulin/protein isolate contains about 5% by weight IgM.

In one embodiment of the invention, the immunoglobulin/protein isolate contains from about 1% to about 10% by weight albumin. In further embodiments the SBI contains from about 1% to about 2%, from about 1% to about 3%, from about 1% to about 4%, from about 1% to about 5%, from about 1% to about 6%, from about 1% to about 7%, from about 1% to about 8%, from about 1% to about 9%, from about 2% to about 4%, from about 2% to about 6%, from about 2% to about 8%, from about 5% to about 10%, from about 3% to about 6%, from about 3% to about 9%, or from about 8% to about 10% by weight albumin. In other embodiments of the invention, the immunoglobulin/protein isolate contains about 1%, 2%, 3%, 4%, 5%, 6%, 7%, 8%, 9% or 10% by weight albumin. In a further embodiment, the immunoglobulin/protein isolate contains about 5% by weight albumin.

The immunoglobulin/protein isolate described herein can be provided or administered in powder form, preferably suspended or dissolved in a suitable liquid, such as water, saline, or milk. The globulin concentrate can be combined with a pharmaceutically acceptable carrier such as a suitable liquid vehicle or excipient and an optional auxiliary additive or additives. The liquid vehicles and excipients are conventional and commercially available. Illustrative thereof are distilled water, physiological saline, aqueous solutions of dextrose, and the like.

In general, in addition to the active compounds, the compositions of this invention may contain suitable excipients and auxiliaries which facilitate processing of the active compounds into preparations which can be used pharmaceutically. Oral dosage forms include tablets, dragees, and capsules.

The compositions for use in the present invention are manufactured in a manner which is itself well known in the art. For example, the preparations may be made by means of conventional mixing, granulating, dragee-making, dissolving, and/or lyophilizing processes. The processes to be used will depend ultimately on the physical properties of the ingredients used and the desired form of the end product.

Suitable excipients are, in particular, fillers such as sugars for example, lactose or sucrose, mannitol or sorbitol, cellulose preparations and/or calcium phosphates, for example tricalcium phosphate or calcium hydrogen phosphate, as well as binders such as starch, rice starch, potato starch, gelatin, gum tragacanth, methyl cellulose, hydroxypropylmethylcellulose, sodium carboxymethylcellulose, and/or polyvinylpyrrolidone. If desired, disintegrating agents may be added, such as the above-mentioned starches as well as carboxymethyl starch, cross-linked polyvinyl pyrrolidone, agar, or alginic acid or a salt thereof, such as sodium alginate. Auxiliaries are flow-regulating agents and lubricants, for examples, such as silica, talc, stearic acid or salts thereof, such as magnesium stearate or calcium stearate and/or polyethylene glycol. Dragee cores may be provided with suitable coatings which, if desired, may be resistant to gastric juices.

For this purpose, concentrated sugar solutions may be used, which may optionally contain gum arabic, talc, polyvinyl pyrrolidone, polyethylene glycol, and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethylcellulose phthalate, dyestuffs and pigments may be added to the tablet or dragee coatings, for example, for identification or in order to characterize different combination of compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils, liquid paraffin, or liquid polyethylene glycols. In addition, stabilizers may be added. In addition to administration with conventional carriers, active ingredients may be administered by a variety of specialized delivery drug techniques which are known to those of skill in the art.

### Dosage, Duration, and Subjects

A “therapeutically effective amount” can be, but is not limited, to an amount of immunoglobulin/protein isolate that increases the subject's CD4⁺ T cell count by at least 10%. In another embodiment of the invention, a therapeutically effective amount of immunoglobulin/protein isolate increases the subject's mucosal CD4⁺ lymphocyte densities by at least 50%. In another embodiment of the invention, a therapeutically effective amount of immunoglobulin/protein isolate increases the CD3⁺:CD4⁺ T cell ratio in duodenal tissue in the subject by at least 50%. In another embodiment of the invention, a therapeutically effective amount of immunoglobulin/protein isolate decreases the plasma IL-6 levels in the subject by at least about 50%. In another embodiment of the invention, a therapeutically effective amount of immunoglobulin/protein isolate decreases the number of duodenal crypt cells expressing Ki67 in the subject by at least about 50%. In another embodiment of the invention, a therapeutically effective amount of immunoglobulin/protein isolate increases the CD4⁺:CD8⁺ T cell ratio in the duodenal tissue of the subject by at least about 50%.

The dosage and number of doses (e.g., single or multiple dose) administered to the subject will vary depending upon a variety of factors, including the route of administration, patient conditions and characteristics (sex, age, body weight, health, size), extent of symptoms, concurrent treatments, frequency of treatment and the effect desired, and the like. These parameters can be determined for each system by well-established procedures and analyses, e.g., in phase I, II, and III clinical trials. In one embodiment of the invention, immunoglobulin/protein isolate is administered once daily. In another embodiment of the invention, immunoglobulin/protein isolate is administered twice daily. In another embodiment of the invention, immunoglobulin/protein isolate is administered three times a day. In another embodiment of the invention, immunoglobulin/protein isolate is administered four times daily. In one embodiment of the invention, immunoglobulin/protein isolate is administered from about 1 week to about 25 weeks. In a further embodiment, immunoglobulin/protein isolate is administered from about 1-4 weeks, 1-5 weeks, 1-10 weeks, 1-15 weeks, 1-20 weeks, 5-10 weeks, 5-15 weeks, 5-20 weeks, 5-25 weeks, 10-15 weeks, 10-20 weeks, 10-25 weeks, 15-20 weeks, or 15-25 weeks. In another embodiment of the invention, immunoglobulin/protein isolate is administered for about 1, 2, 3, 4, 5, 6, 7, 8, 9, or 10 months. In another embodiment of the invention, immunoglobulin/protein isolate is administered from about 1-2 months, 1-3 months, 1-4 months, 1-5 months, 1-6 months, 1-7 months, 1-8 months, 1-9 months, 1-10 months, 2-3 months, 2-4 months 2-5 months, In a further embodiment, SBI is administered for about 1 week. In a further embodiment, immunoglobulin/protein isolate is administered for about 2 weeks. In a further embodiment, SBI is administered for about 3 weeks. In a further embodiment, immunoglobulin/protein isolate is administered for about 4 weeks. In a further embodiment, the immunoglobulin/protein isolate is administered for about 24 weeks. In yet a further embodiment, the immunoglobulin/protein isolate is administered twice daily for about 24 weeks.

In one embodiment of the invention, the immunoglobulin/protein isolate is administered in an amount from about 0.5 g to about 15 g to the subject daily. In a further embodiment of the invention, the amount of immunoglobulin/protein isolate administered to the subject daily is from about 1 g to about 15 g, from about 1 g to about 10 g, from about 1 g to about 5 g, from about 2 g to about 5 g, from about 2 g to about 10 g, from about 2 g to about 15 g, from about 5 g to about 10 g, from about 5 g to about 15 g, or from about 10 g to about 15 g. In another embodiment of the invention, the amount of SBI administered to the subject daily is about 1 g, 2 g, 3 g, 4 g, 5 g, 6 g, 7 g, 8 g, 9 g, 10 g, 11 g, 12 g, 13 g, 14 g, or 15 g. In a further embodiment, about 2.5 g of immunoglobulin/protein isolate is administered to the subject twice daily. In a further embodiment, about 5 g of immunoglobulin/protein isolate is administered to the subject twice daily.

In one embodiment of the invention, the immunoglobulin/protein isolate is administered in an amount from about 0.05% to about 5% by weight of the subject's total daily dietary intake. In a further embodiment of the invention, the immunoglobulin/protein isolate is administered in an amount from about 0.05% to about 0.1%, from about 0.05% to about 0.2%, from about 0.05% to about 0.5%, from about 0.05% to about 1% from about 0.05% to about 2%, from about 0.1% to about 0.2%, from about 0.1% to about 0.5%, from about 0.1% to about 1% from about 0.1% to about 2% from about 0.1% to about 5%, from about 0.5% to about 1%, from about 0.5% to about 2%, from about 0.5% to about 5%, from about 1% to about 2%, from about 1% to about 5%, or from about 2% to about 5% by weight of the subject's total daily dietary intake. In another embodiment, the immunoglobulin/protein isolate is administered in an amount of about 0.05%, 0.1%, 0.2%, 0.5%, 1%, 1.5%, 2%, 2.5%, 3%, 4%, or 5% by weight of the subject's total daily dietary intake. In a further embodiment, the amount of immunoglobulin/protein isolate administered is about 0.2% by weight of the subject's total daily dietary intake. In a further embodiment, the amount of immunoglobulin/protein isolate administered is about 0.4% by weight of the subject′ total daily dietary intake.

“Administering” a composition may be accomplished by oral administration, injection, infusion, parenteral, intravenous, mucosal, sublingual, intramuscular, intradermal, intranasal, intraperitoneal, intraarterial, subcutaneous absorption or by any method in combination with other known techniques. In one embodiment of the invention, the immunoglobulin/protein isolate is administered orally.

The term “subject” as used herein is limited to humans. In some embodiments, the human subject is one who exhibits virologic suppression for at least 48 weeks. Virologic suppression is defined as plasma HIV RNA quantities in a subject below the limit of detection. Detection limits may vary depending on the assay used. For example, the Roche Amplicor HIV-1 Monitor Assay has a detection limit of <50 copies/mL. The Versant HIV-1 RNA Assay has a detection limit of <75 copies/mL. The Abbott m2000sp/m2000rt real-time PCR Test has a detection limit of <40 copies/mL. Finally, the COBAS AmpliPrep/TAQMAN HIV-1 Assay has a detection limit of <48 copies/mL. In some embodiments, the human subject is one who has a baseline HIV viral load of from about 20 to about 100 copies/mL. In one embodiment of the invention, subject has a baseline HIV viral load of from about 20 to about 40 copies/mL. In further embodiments, the subject has a baseline HIV viral load of from about 20 to about 25, from about 20 to about 30, from about 20 to about 35, from about 25 to about 30, from about 25 to about 35, from about 25 to about 40, from about 30 to about 35, from about 30 to about 40, or from about 35 to about 40 copies/mL.

In some embodiments, the human subject is one who has been on ART for at least about 48 weeks. In other embodiments, the human subject has been on ART for about 48 weeks to about 15 years. In further embodiments, the human subject has been on ART from about 48 weeks to about 1 year, from about 1 year to about 2 years, from about 2 years to about 5 years, from about 48 weeks to about 2 years, from about 48 weeks to about 5 years, from about 5 to about 10, from about 10 to about 15, or from about 8 to 10 years. In another embodiment, the human subject has been on ART for about 5, 6, 7, 8, 9, 10, 11, 12, 13, 14, or 15 years. In another embodiment, the human subject has been on ART for about 48 weeks, 49 weeks, 50 weeks, 51 weeks, 1 year, 2 years, 3 years, or 4 years. In a further embodiment, the human subject has been on ART for about 5 years.

In some embodiments, the human subject is one who has been infected with HIV from about 5 to about 15 years. In other embodiments, the human subject has been infected with HIV from about 10 to 15, from about 5 to 10, or from about 8 to about 10 years. In another embodiment of the invention, the human subject has been infected with HIV for about 5, 6, 7, 8, 9, 10, 11, 12, 13, 14, or 15 years. In a further embodiment, the human subject has been infected with HIV for about 15 years.

In some embodiments, the human subject has experienced HIV-associated enteropathy for at least about 5 years. In other embodiments, the human subject has experienced HIV-associated enteropathy from about 5 to about 15 years. In further embodiments, the human subject has experienced HIV-associated enteropathy from about 5 to about 10, from about 10 to about 15, or from about 8 to 10 years. In another embodiment, the human subject has experienced HIV-associated enteropathy for about 5, 6, 7, 8, 9, 10, 11, 12, 13, 14, or 15 years. In a further embodiment, the human subject has experienced HIV-associated enteropathy for about 5 years.

In some embodiments, the human subject has a peripheral CD4⁺ T cell count of less than about 500 cells/mm³ at baseline. In other embodiments, the human subject has a peripheral CD4⁺ T cell count of from about 300 to about 400 cells/mm³ at baseline. In further embodiments, the human subject has a peripheral CD4⁺ T cell count of from about 300 to about 400, from about 300 to about 325, from about 300 to about 350, from about 300 to about 375, from about 325 to about 350, from about 325 to about 375, from about 325 to about 400, from about 350 to about 375, from about 350 to about 400, from about 375 to about 400, from about 300 to about 500, from about 400 to about 500, from about 450 to about 500, from about 475 to about 500, from about 400 to about 425, from about 425 to about 500, from about 425 to about 450, or from about 400 to about 450 cells/mm³ at baseline.

The term “HIV-associated enteropathy” can include, but is not limited to, the following symptoms: diarrhea, increased GI inflammation, increased intestinal permeability, (up to fivefold higher than in healthy controls), malabsorption of bile acid and vitamin B12. As discussed in J. M. Brenchley & D. C. Douek, 1(1) MUCOSAL IMMUNOL. 23-30 (2008), the enteropathy involves inflammatory infiltrates of lymphocytes and damage to the GI epithelial layer, including villous atrophy, crypt hyperplasia, and villous blunting. Brenchley and Douek note that these pathologic changes occur in the absence of the detectable bacterial, viral, or fungal enteropathogens that are often associated with enteropathy.

Having described the invention with reference to particular compositions, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

### Example 1

### SBI Increases Peripheral and Mucosal CD4⁺ T Cell Count

A multi-center trial in HIV-associated enteropathy was conducted to evaluate the impact of serum-derived immunoglobulin concentrate, specifically SBI, on markers of peripheral and mucosal immunity.

### Methods

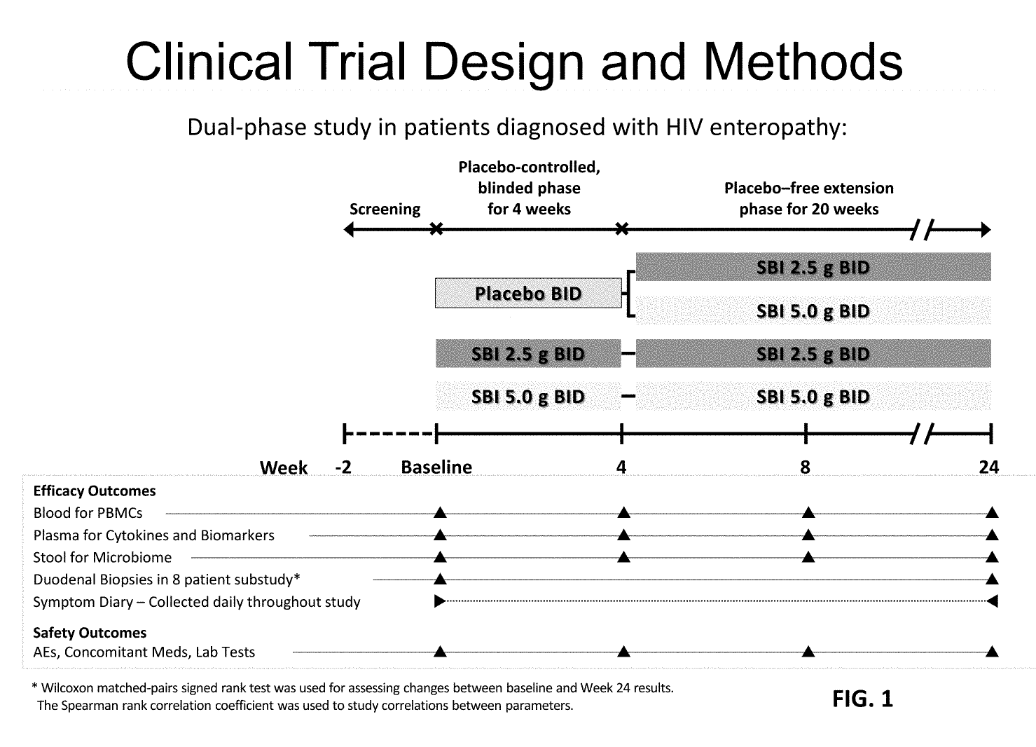

Participants were diagnosed with HIV-associated enteropathy after an extensive negative work-up for gastrointestinal (GI) disease and were on chronic suppressive ART. HIV-associated enteropathy was defined as abnormal passage of 3 or more unformed stools per day for at least 4 weeks duration. Patients were randomized to receive 2.5 g of SBI twice-daily (SBI 2.5 g BID), 5.0 g of SBI twice-daily (SBI 5.0 g BID), or placebo (PBO) during a 4 week cross-over lead-in phase for safety evaluation, and then continued on open-label for 20 weeks. Evaluation included patient-reported surveys on GI symptoms and other health parameters, and change in nutrient absorption and plasma levels of CD4⁺ and CD8⁺ lymphocyte numbers, enterocyte damage biomarkers, and pro-inflammatory cytokines. Wilcoxon matched-pairs signed rank test was used for assessing changes from baseline results. See FIG. 1 regarding the clinical trial design and schedule of outcome procedures.

### Results

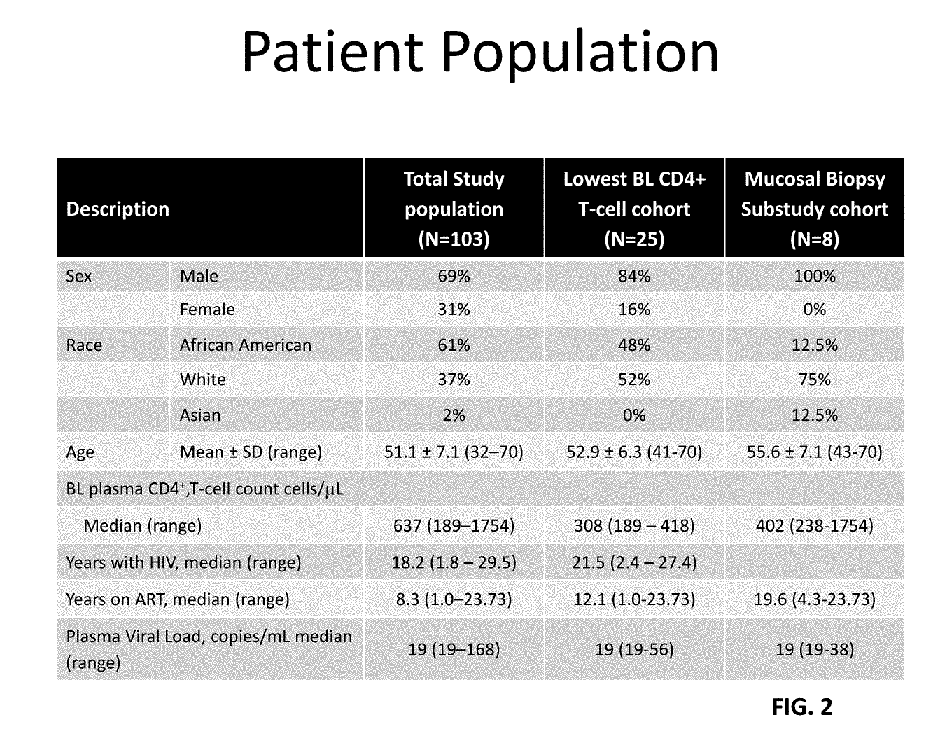

103 patients (N=34, 2.5 g SBI BID; N=33, 5.0 g SBI BID; N=36, PBO) were enrolled (69% male; 61% black; median age of 50 years) with a mean duration of HIV, ART, and enteropathy over 16.9, 11.3, and 5.6 years, respectively. The majority of patients had a baseline HIV viral load <40 copies/mL with a median peripheral CD4⁺ T cell count of 637 cells/mm³. Many of these patients also had a plasma HIV viral load ≦40 copies/mL for at least 48 weeks prior to screening/baseline. See FIG. 2 regarding the patient population.

All participants, regardless of treatment group, showed a significant reduction in frequency of abnormal stools (p≦0.001) from baseline at week 4, and between group analysis was not significant at week 4.

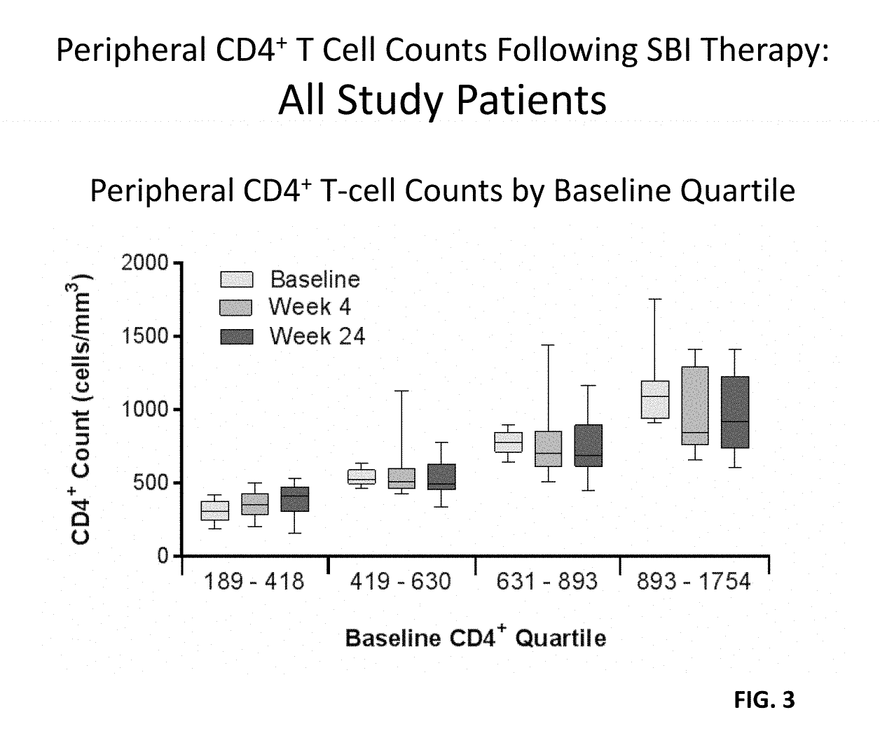

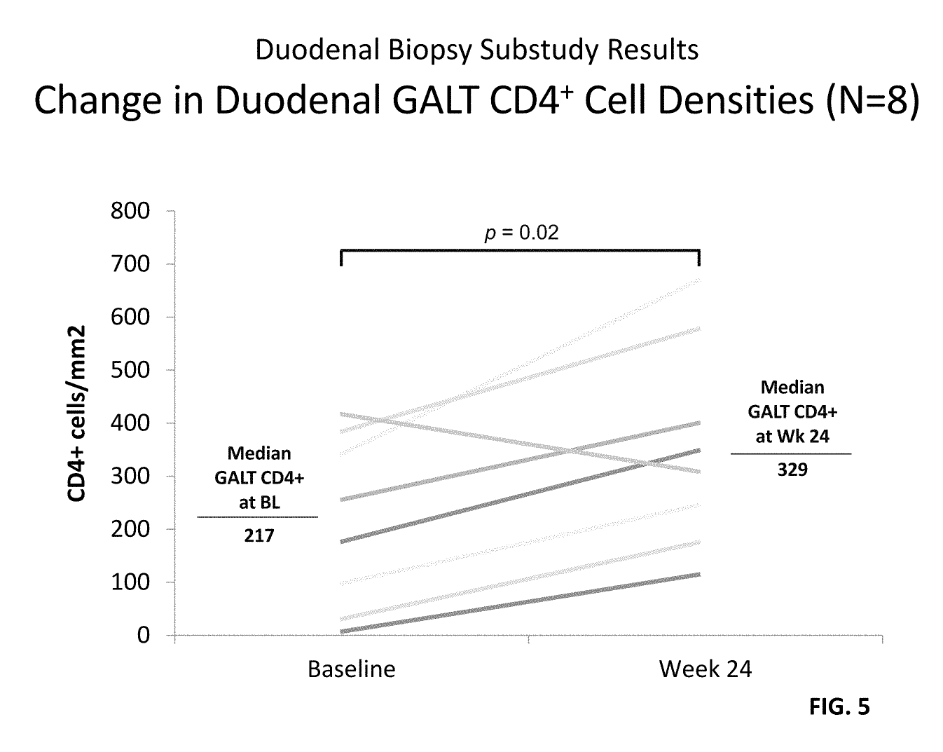

Following 24 weeks of therapy, there were observable changes from baseline in peripheral CD4⁺ T cells for the SBI groups which varied depending on the baseline CD4⁺ T cell count of the subject. FIG. 3 illustrates the change in peripheral CD4⁺ T cell counts of study participants after 4 and 24 weeks of SBI treatment broken down by baseline CD4⁺ T cell quartile, while FIG. 5 focuses on the peripheral CD4⁺ T cell counts of study participants in the lowest baseline CD4⁺ T cell quartile. Patients (N=24) in the bottom quartile, who had a baseline peripheral CD4⁺ T cells≦418 cells/mm³, demonstrated an increase of 42 cells/mm³ in peripheral CD4⁺ T cells after 4 weeks of SBI treatment and an increase of 95 cells/mm³ in peripheral CD4⁺ T cells after 24 weeks of SBI treatment, both as compared to baseline CD4⁺ T cell count. In contrast, the 4 week placebo-administered group showed a decrease in CD4⁺ T-cell count as compared to baseline. The increase in the 4 and 24 week SBI groups were statistically significant in comparison to the placebo group (p=0.02 and p=0.007, respectively). Patients (N=24) in the bottom quartile (having baseline peripheral CD4⁺ T cells≦418 cells/mm³) also exhibited a decrease in mean plasma IL-6 at 24 weeks (0.91±0.94 pg/mL) as compared to baseline measurements (2.39±3.02 pg/mL). Plasma IL-6 was measured using the Magnetic Luminex Performance Assay and Human IL-6 High Sensitivity Kit.

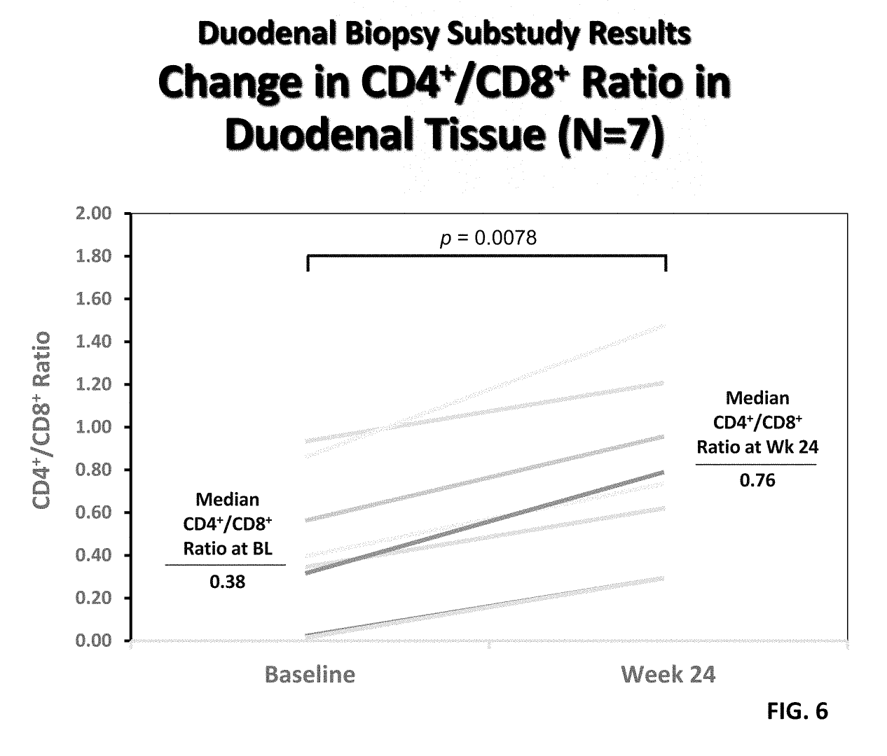

Additionally, as shown in FIG. 5, biopsies obtained from patients receiving the SBI for 24 weeks (N=8) revealed that the median duodenal GALT CD4⁺ T cell density increased by 145 cells/mm² [p=0.102] from 217 to 329 cells/mm² (as compared to baseline level) throughout the time course of treatment. Similarly, as shown in FIG. 6, the median CD4⁺/CD8⁺ ratio significantly (p=0.0078) increased from 0.38-0.76 (as compared to baseline level)_throughout the time course of treatment in patients receiving SBI (N=7). For immunofluorescence analysis, the primary antibodies used were polyclonal anti-CD3 rabbit serum (Dako Inc., Carpinteria, Calif., USA) and monoclonal anti-CD4 or CD8 mouse serum (Leica Microsystems, Buffalo Grove, Ill., USA). Binding of CD3⁺ and CD4⁺ or CD8⁺ receptors was detected simultaneously using Alexafluor488-labeled polyclonal goat anti-rabbit IgG and Alexafluor568-labeled polyclonal goat anti-mouse IgG (both Molecular Probes, Eugene, Oreg., USA). Positive cells were counted by a single observer (Z.-M.M.) and presented as cells/mm² of lamina propria or intraepithelial regions of duodenal mucosa.

Further, duodenal crypt cells expressing Ki67 significantly (p=0.08) decreased (as compared to baseline level) in patients receiving SBI for 24 weeks (N=7) from 40.5% to 24.4%, which also correlated with a significantly (p=−0.048) decreased number of Paneth cells per crypt in patients receiving SBI for 24 weeks (N=8). These results are shown in FIG. 8. Briefly, for counting Ki67 cells, monoclonal mouse anti-human Ki67 antibody (BD Biosciences, San Jose, Calif., USA) and DAPI were used to stain slides. Crypts with complete structure (showing bottom, body and mouth) were chosen for study. Total cells number was noted by counting nuclei stained by DAPI. Ki67 positive cells were counted based on Ki67 staining. Results of Ki67 positive cells were reported as the percentage of Ki67 cells over total cells of the chosen crypts. The number of Paneth cells in crypts was generated by reading H.E. slides of biopsies. Results were reported as the number of Paneth cells per crypts.

### CONCLUSIONS

Orally administered SBI was well tolerated by patients receiving ART and was found to significantly increase peripheral and intestinal mucosal CD4⁺ T cell counts leading to immune reconstitution. These results demonstrate that oral SBI is an effective therapy for restoring mucosal immunity and reconstituting systemic immune function among patients who have not achieved normal CD4⁺ T cell counts despite prolonged suppressive ART.

1. A method of treating human immunodeficiency virus (HIV)-associated enteropathy in a subject, comprising:
(1) identifying a subject who has a baseline HIV viral load of from about 20 to about 100 copies/mL and has been on antiretroviral therapy (ART) for at least about 48 weeks; and
(2) orally administering to the subject a therapeutically effective amount of immunoglobulin/protein isolate, wherein the immunoglobulin/protein isolate contains at least about 60% by weight immunoglobulin (Ig).

2. The method of claim 1, wherein the immunoglobulin/protein isolate is obtained from bovine, ovine, or porcine blood, plasma, or serum, or a combination thereof.

3. The method of claim 2, wherein the immunoglobulin/protein isolate comprises serum-derived bovine immunoglobulin/protein isolate (SBI).

4. The method of claim 1, wherein the immunoglobulin/protein isolate contains from about 60% to about 80% by weight Ig.

5. (canceled)

6. The method of claim 1, wherein the immunoglobulin/protein isolate contains greater than about 50% by weight IgG and less than about 80% by weight IgG.

7. The method of claim 1, wherein the immunoglobulin/protein isolate contains from about 1% to about 10% by weight IgA.

8. (canceled)

9. The method of claim 1, wherein the immunoglobulin/protein isolate contains from about 1% to about 10% by weight IgM.

10. (canceled)

11. The method of claim 1, wherein the immunoglobulin/protein isolate contains from about 1% to about 10% by weight albumin.

12. (canceled)

13. The method of claim 1, wherein the subject has a baseline HIV viral load of from about 20 to about 40 copies/mL.

14. The method of claim 1, wherein the subject has a peripheral CD4⁺ T cell count of less than about 500 cells/mm³ at baseline.

15. (canceled)

16. The method of claim 1, wherein from about 0.5 g to about 15 g of immunoglobulin/protein isolate is administered to the subject daily.

17-18. (canceled)

19. The method of claim 1, wherein the amount of immunoglobulin/protein isolate administered is from about 2.5% to about 30% of the subject's total daily dietary protein intake.

20. (canceled)

21. The method of claim 19, wherein the amount of immunoglobulin/protein isolate administered is from about 5% to about 20% of the subject's total daily dietary protein intake.

22. The method of claim 1, wherein the immunoglobulin/protein isolate is administered for about 1 week to about 25 weeks.

23-27. (canceled)

28. The method of claim 1, wherein the subject's peripheral CD4⁺ T cell count increases by at least about 10%.

29. The method of claim 1, wherein the subject's mucosal CD4⁺ lymphocyte densities increases by at least about 50%.

30. (canceled)

31. The method of claim 1, wherein the plasma IL-6 levels in the subject decreases by at least about 50%.

32. The method of claim 1, wherein the duodenal crypt cells expressing Ki67 in the subject decrease by at least about 50%.

33. The method of claim 1, wherein the CD4⁺:CD8⁺ T cell ratio in the duodenal tissue of the subject increases by at least about 50%.
